(19) United States
(12) Patent Application Publication
(10) Pub. No.: US 2025/0195271 A1
(43) Pub. Date: Jun. 19, 2025

(54) OPHTHALMIC BLADE WITH MOVEABLE SECTION

(71) Applicant: Ophthalmic Neo-Innovations of Texas, LLC, Sugar Land, TX (US)

(72) Inventor: John Morgan Micheletti, Sugar Land, TX (US)

(21) Appl. No.: 19/064,864

(22) Filed: Feb. 27, 2025

Related U.S. Application Data
(63) Continuation of application No. 18/486,154, filed on Oct. 12, 2023.
(60) Provisional application No. 63/415,657, filed on Oct. 13, 2022.
(60) Provisional application No. 63/559,624, filed on Feb. 29, 2024.

(51) Int. Cl. A61F 9/007; A61B 17/3211

(52) U.S. Cl. CPC A61F 9/00754; A61B 2017/32113

(57) ABSTRACT

An ophthalmic instrument includes a blade having an anterior blade portion that is moveable relative to a fixed blade portion, the anterior having a triangular head having opposed anterior cutting edges and the fixed blade portion having opposed posterior cutting edges where anterior blade portion is moveable between a retracted position where the opposed anterior cutting edges and the opposed posterior cutting edges present substantially continuous cutting edges, and an extended position where the triangular head is spaced forward of the posterior cutting edges.

### TECHNICAL FIELD

This disclosure relates in general to the field of surgical instruments for ophthalmological procedures for treatment of eye diseases, such as cataract, and more particular to a blade for incorporating for making corneal incisions.

### BACKGROUND

This section provides background information to facilitate a better understanding of the various aspects of the disclosure and is not an admission of prior art.

Cataract surgery is the most commonly performed surgery in the United States and results with removal of a cataract and implantation of an intraocular lens (IOL). Nearly 3-4 milling people a year have cataract surgery and the number continues to increase. There is a significant amount of waste generated from cataract surgery due to the multiple single-use instruments required for each procedure.

### SUMMARY

An exemplary ophthalmic instrument includes a blade having an anterior blade portion that is moveable relative to a fixed blade portion, the anterior having a triangular head having opposed anterior cutting edges and the fixed blade portion having opposed posterior cutting edges where anterior blade portion is moveable between a retracted position where the opposed anterior cutting edges and the opposed posterior cutting edges present substantially continuous cutting edges, and an extended position where the triangular head is spaced forward of the posterior cutting edges.

An exemplary ophthalmic instrument includes a blade extending in a horizontal plane along a central longitudinal axis and a lateral axis orthogonal to the longitudinal axis, the blade symmetric about the central longitudinal axis. The blade includes an anterior blade portion having a triangular head and a shaft. The triangular head has opposed anterior cutting edges extending from an anterior tip to a base position that has a base width parallel with the lateral axis. The shaft extends aft of the base position and has a shaft width between opposed shaft edges. A fixed blade portion has a forward lateral position with a forward width and a posterior lateral position with a posterior width that is greater than the forward width and opposed posterior cutting edges extending from the forward lateral position to the posterior lateral position. A pocket is formed in the fixed blade portion and extends aft along, and bisected by, the central longitudinal axis where the shaft is positioned in the pocket and moveable between a retracted position with the base position located with the forward lateral position whereby the opposed anterior cutting edges and the opposed posterior cutting edges present substantially continuous cutting edges, and an extended position where the base position is spaced forward of the forward lateral position by an extension length. In some embodiments, the base width of the triangular head is equal to the shaft width. In another embodiments, the base width of the triangular head is greater than the shaft width.

The ophthalmic instrument may further include a guard having a first leg generally orthogonal to a second leg, wherein in use and in a partial operational position the first leg is attached to the handle, the fixed blade portion is inside of the second leg, and the triangular blade portion and the shaft are positioned outside of the guard. The guard can be moved to a full operational position exposing all of the cutting edges of the blade.

This summary is provided to introduce a selection of concepts that are further described below in the detailed description. This summary is not intended to identify key or essential features of the claimed subject matter, nor is it intended to be used as an aid in limiting the scope of claimed subject matter.

### BRIEF DESCRIPTION OF THE DRAWINGS

The disclosure is best understood from the following detailed description when read with the accompanying figures. It is emphasized that, in accordance with standard practice in the industry, various features are not drawn to scale. In fact, the dimensions of various features may be arbitrarily increased or reduced for clarity of discussion. As will be understood by those skilled in the art with the benefit of this disclosure, elements and arrangements of the various figures can be used together and in configurations not specifically illustrated without departing from the scope of this disclosure.

FIG. 1 illustrates an example ophthalmic instrument with a multifunction ophthalmic blade according to one or more aspects of the disclosure.

FIG. 2 illustrates an example ophthalmic instrument in the form of a multifunction ophthalmic blade according to one or more aspects of the disclosure.

FIG. 3 illustrates an example with ophthalmic instrument in a full operational position with an example guard removed from attachment with the instrument according to one or more aspects of the disclosure.

FIG. 4 illustrates an example with ophthalmic instrument in a full operational position with an example guard attached to the instruments handle and moved to a position exposing all cutting edges of the blade for creating an incision.

FIG. 5 illustrates an example with ophthalmic instrument in a partial operational position with an example guard covering aft cutting edges and exposing front cutting edges for creating an incision with the forward cutting edges.

FIG. 6 illustrates an example multifunction ophthalmic blade constructed of two or more separate pieces of material.

FIG. 7 illustrates an example multifunction ophthalmic blade constructed of two separate pieces of material.

FIG. 8 illustrates an example multifunction ophthalmic blade constructed of two separate pieces of material with an anterior piece removed.

FIG. 9 illustrates another example multifunction ophthalmic blade constructed of two separate pieces of material with an anterior piece removed.

FIG. 10 is a diagrammatic sectional view of a human eye.

FIG. 11 is front end view of an example fixed blade portion of ophthalmic blade for combination with a moveable anterior blade portion.

FIG. 12 is a top view of the example fixed body portion of FIG. 11.

FIG. 13 illustrates an example of a moveable anterior blade portion for use with the fixed body portion of FIGS. 11 and 12.

FIG. 14 illustrates an example ophthalmic blade, assembled from the combination of FIGS. 11-13, with the moveable blade portion in a retracted position.

FIG. 15 illustrates the example ophthalmic blade of FIG. 14 with the moveable blade portion in an extended position.

FIG. 16 is a front view of another example of fixed blade portion with a pocket configured to dispose the moveable blade portion of FIG. 17.

FIG. 17 is an example of a moveable blade portion for combination with the fixed blade portion of FIG. 16.

FIG. 18 is front end view of another example fixed blade portion of ophthalmic blade configured to accept a moveable blade portion.

FIG. 19 is a top view of the fixed blade portion of FIG. 18.

FIG. 20 illustrates an example of a moveable anterior blade portion, with an arrowhead shape, for use with the fixed body portion of FIGS. 18 and 19.

FIG. 21 illustrates an example ophthalmic blade, assembled from the combination of FIGS. 18-20, with the moveable blade portion in a retracted position.

FIG. 22 illustrates the example ophthalmic blade of FIG. 21 with the moveable blade portion in an extended position.

FIG. 23 is a front view of another example of fixed blade portion with a pocket configured to dispose the moveable blade portion of FIG. 24.

FIG. 24 is an example of a moveable blade portion for combination with the fixed blade portion of FIG. 23.

FIG. 25 illustrates an example ophthalmic blade and actuator to move the moveable blade portion.

FIG. 26 illustrates another example ophthalmic blade and actuator to move the moveable blade portion.

FIG. 27 is a schematic illustration of an actuator coupled to a moveable blade portion of an example ophthalmic blade.

### DETAILED DESCRIPTION

It is to be understood that the following disclosure provides many different embodiments, or examples, for implementing different features of various illustrative embodiments. Specific examples of components and arrangements are described below to simplify the disclosure. These are, of course, merely examples and are not intended to be limiting. For example, a figure may illustrate an exemplary embodiment with multiple features or combinations of features that are not required in one or more other embodiments and thus a figure may disclose one or more embodiments that have fewer features or a different combination of features than the illustrated embodiment. Embodiments may include some but not all the features illustrated in a figure and some embodiments may combine features illustrated in one figure with features illustrated in another figure. Therefore, combinations of features disclosed in the following detailed description may not be necessary to practice the teachings in the broadest sense and are instead merely to describe particularly representative examples. In addition, the disclosure may repeat reference numerals and/or letters in the various examples. This repetition is for the purpose of simplicity and clarity and does not itself dictate a relationship between the various embodiments and/or configurations discussed.

Cataract surgery requires multiple incisions in the cornea, most commonly consisting of a smaller “side port” incision of approximately 1 mm and a main “wound” consisting of a 1.4 mm to 3.2 mm incision. Additionally, an instrument called a cystotome is used to enter the anterior capsule. Each of these incisions are performed with a different single use instrument. There currently exists no option for a single instrument to create both corneal incisions of varying sizes and the initial incision into the anterior capsule. Embodiments of the ophthalmic blade address all three of these functions in a single device, which improves surgical efficiency and reduces waste.

The multifunctional blade is described with reference to a three mutually orthogonal directions X, Y, and Z forming a three-dimensional frame of reference XYZ. Longitudinal axis X corresponds to the axis that extends through the center of the blade in the fore and aft directions. Transverse or lateral axis Y is perpendicular to longitudinal axis X and corresponds to the width of the blade. The X-Y plane is considered to be a horizontal plane. Vertical axis Z is oriented perpendicular with respect to the X-Y plane.

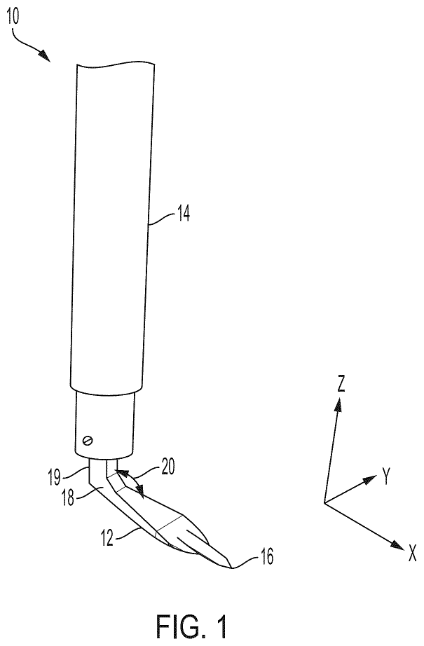

FIG. 1 illustrates an exemplary embodiment of an ophthalmic instrument, e.g., a surgical knife, generally denoted by the numeral 10. Instrument 10 includes a blade 12 that in use is attached to a handle 14. Blade 12 generally extends in a horizontal plane (X-Y plane) from an anterior tip 16 to an aft elbow 18. In this example elbow 18 includes an arm 19 that extends to handle 14 at an elbow angle 20 of about 90 degrees to the horizontal plane. Blade 12 may be removably attached via various mechanism so that the blade can be removed and the handle can be reused. In some embodiments, elbow angle 20 may be 180 degrees, such that the handle is parallel with blade. Elbow angle 20 is generally between 0 and 180 degrees. Blade 12 may be constructed of various materials of construction including steel or alloys for example as disposable blades or more resistant materials such as diamond or sapphire which can be reused.

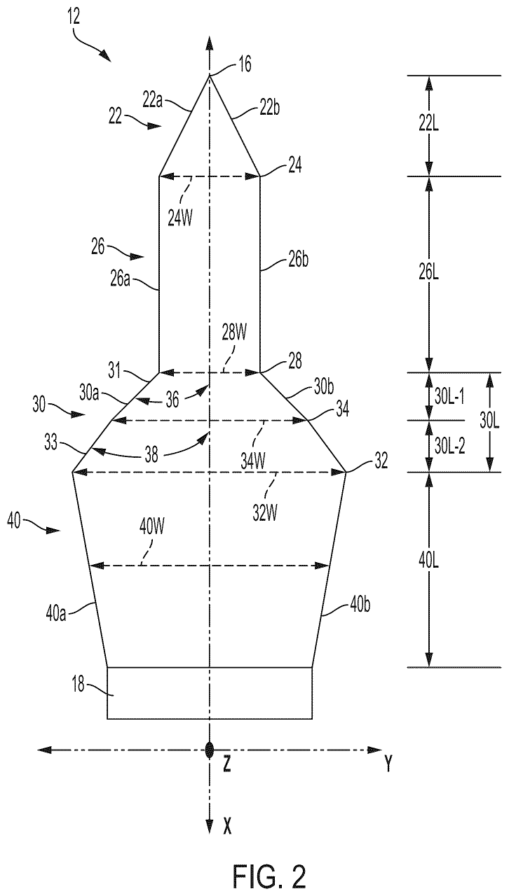

FIG. 2 illustrates an exemplary blade 12 extending in a horizontal plane (X-Y plane) along a longitudinal axis “X” and a lateral axis “Y” orthogonal to the longitudinal axis, the blade is laterally symmetric about the longitudinal axis. Blade 12 has cutting edges on sections of the opposed lateral sides of the blade. Blade 12 includes a central and anterior triangular portion 22 having opposed, first or anterior, cutting edges 22a, 22b and a first longitudinal length 22L extending from anterior tip 16 to a first position 24. Cutting edges 22a, 22b extend from anterior tip 16 to first position 24 and define anterior triangular portion 22. First position 24 extends laterally to the terminal ends of cutting edges 22a, 22b and has a first position width 24W parallel to the lateral axis Y. A second or longitudinal shaft portion 26 has parallel edges 26a, 26b and extends a second longitudinal length 26L from first position 24, the base of triangular portion 22, to a second position 28 located on a third blade portion 30 having opposed second cutting edges 30a, 30b. Second position 28 has a lateral width 28W extending laterally to the leading ends of second cutting edges 30a, 30b. Cutting edges 30a, 30b extend at the leading end from second position 28 to a terminal end at a third position 32 on third blade portion 30. Third position 32 has a lateral width 32W that is greater than second position width 28W and first position width 22W. It will be recognized with benefit of this disclosure that blade 12 may be constructed of a single, continuous piece of material or constructed of two or more pieces of material that may be interconnected to form blade 12 prior to being connected to a handle or interconnected by the connection of the pieces to the handle.

In some configurations, first (triangular base) width 24W and second width 28W are equal. In some embodiments, first triangular portion 22 may be arrowhead shaped relative to second portion 26 with first width 24W greater than second width 28W.

In some embodiments, such as illustrated in FIG. 2, cutting edges 30a, 30b do not extend at a constant angle from second position 28 to third position 32. An angle relative to the longitudinal axis includes an arc. In FIG. 2, cutting edges 30a, 30b extend from second position 28 to an intermediate point, or position, 34 at an anterior angle 36 relative to the longitudinal axis “X” and extends from intermediate point 34 to third position 32 at a posterior angle 38 relative to the longitudinal axis that is different from anterior angle 36. Blade 12 has a third intermediate width 34W. The first or anterior leg 31 of third opposed cutting edges 30a, 30b has a longitudinal length of 30L−1 and the second or posterior leg 33 of third opposed cutting edges 30a, 30b has a longitudinal length of 30L−2.

In this embodiment, blade 12 has a terminal portion 40 that extends longitudinally away from third position 32 and anterior tip 16 toward elbow 18. The opposed terminal edges 40a, 40b taper inward (toward center axis X) so that a terminal width 40W of the terminal portion narrows from third width 32W. Terminal portion 40 extends a longitudinal length 40L for example to elbow 18.

In a non-limiting example, first length 22L and the first width 24W are each about 1 mm, second longitudinal length 26L is about 2 mm, third width 32W is about 2.0 mm to 3.4 mm, and third length 30L is about 1 mm. When applicable, length 30L-1 and 30L-2 may be equal, for example, about 0.5 mm when third length 30L is about 1 mm. In a non-limiting example, intermediate width 34W is about 2.0 mm and third width 32W is about 2.4 mm where the first and second widths are each about 2 mm.

First width 24W may be in the range, for example, of about 0.8 mm to 1.4 mm. First width 24W may be less than 1 mm or greater than 1 mm. The largest width, third width 32W may be for example in the range of about 1.4 mm to 3.4 mm. In another example, the third width may be in the range of about 2.0 to 3.4 mm. In another example, the third width is in the range of about 2.0 mm to 3.2 mm.

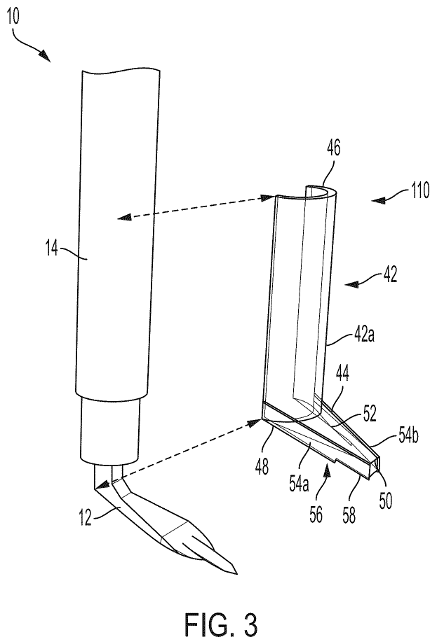

Embodiments and methods disclosed herein utilize a new guard. FIGS. 3-5 illustrate an exemplary guard 40. Guard 40 is configured for moveable connection with an ophthalmic instrument 10. Guard 40 includes a first leg 42 and a second leg 44. First leg 42 is configured to attach to handle 14 and be moveable relative to the handle. First leg 42 may be moveable along the length of the handle or removable from the handle. The illustrated first leg 42 is a partial, semi-circular, cylinder for snapping on to handle 14. First leg 42 is an elongate cylinder or semi-cylinder extending from a top end 46 to a bottom end 48. Second leg 44 extends away from a front side 42a of first leg 42 to a front end 50. Front end 50 is open to pass triangular portion 22 and second portion 26 when guard 40 is in a partial operational position (FIG. 5). Second leg 44 has a top side 52 and opposed sides 54a, 54b formed of the material of construction and extending to front end 50. Forming an internal cavity 56 that is open at a bottom side 58 of second leg 44.

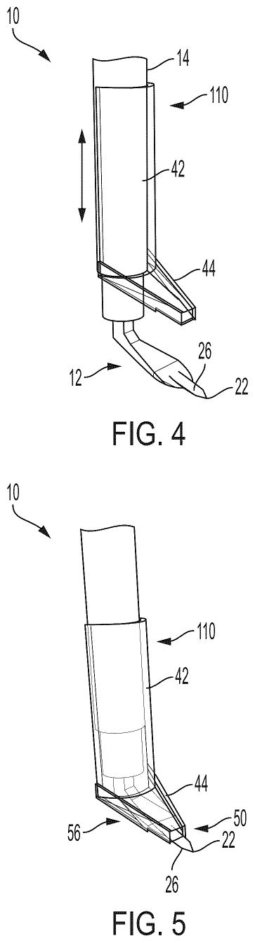

FIG. 4 illustrates guard 110 positioned on the handle 14 of instrument 10. First leg 42 is attached to handle 14 and guard 110 is axially moveable on handle 14. In the full operational position blade 12 is completely removed from second leg 44 whereby each of the cutting edges for blade 12 can be used. It should be recognized that the full operational position can be achieved by removal of guard 110 from instrument 10 as shown in FIG. 3.

FIG. 5 illustrates guard 110 in a partial operational position. In the partial operational position, the aft portion of blade 12 is positioned inside of second leg 44 with second portion 26 and first triangular portion 22 extending through front end 50 and exposed for use to create an incision in a cornea.

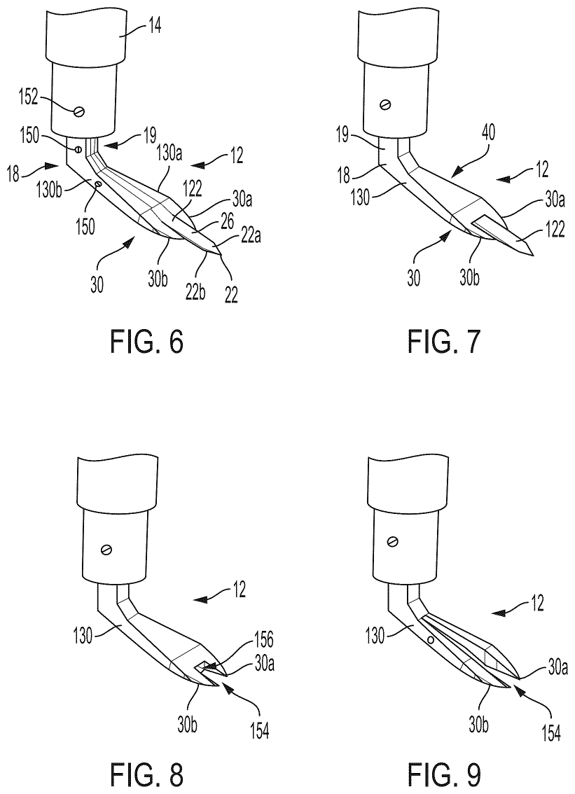

As previously noted, blade 12 may be formed by a single, continuous piece of material, or two or more pieces that are interconnected to provide the stepped shape and different cutting edges. FIGS. 6-9 illustrate exemplary aspects of blades 12 formed of two or more pieces of material, which are described with reference to FIGS. 1-2. Using two or more blade pieces with the cutting edges on different pieces may promote more efficient and effective sharpening of the cutting edges.

FIG. 6 illustrates a blade 12 formed of two or more pieces of material that in a fixed position relative to each other. Blade 12 includes an anterior piece 122 of material carrying first triangular portion 22 with first opposed cutting edges 22a, 22b, and second shaft portion 26. Anterior piece 122 cooperates with and is interconnected with one or more lateral pieces of material carrying third portion 30 and cutting edges 30a, 30b. For example, in FIG. 6 blade 12 includes a first lateral piece 130a having third cutting edge 30a and a second lateral piece 130b. Lateral pieces 130a, 130b, and anterior piece 122 are aligned to form the desired multiple cutting edge blade 12. Anterior piece 122 and lateral pieces 130a, 130b may each include a portion of elbow 18 and arm 19. The blade pieces may be interconnected by various fasteners including without limitation screws, pins, welds, solder, friction fit, and adhesives. For example, FIG. 6 illustrates blade fasteners 150 in the form of screws or pins that hold the blade pieces 130a, 122, 130b pieces together as complete blade 12 even when blade 12 is not connected to handle 12. Blade 12 is illustrated as attached via arm 19 to handle 14 via handle fastener 152 which again is shown in the form of a screw or pin that facilitates detaching blade 12 from the handle. Handle fastener 152 may serve as the fastener that fixedly interconnects the blade pieces together in the multiple cutting edge blade configuration.

FIGS. 7-9 illustrate exemplary aspects of blade 12 constructive of two separate pieces of material, anterior piece 122 including the first triangular portion and the second portion and a lateral piece 130 that includes third portion 30, third opposed cutting edges 30a, 30b, terminal portion 40, elbow 18 and arm 19.

Anterior piece 122 is removed in FIGS. 8-9 illustrating lateral piece 130 alone. In each of FIGS. 8 and 9, lateral piece 130 creates an opening 154 between third opposed cutting edges 30a, 30b in which the anterior portion is to be positioned. In FIG. 8, lateral piece 130 may form a cavity 156 between edges 30a, 30b into which a portion of the anterior piece can be inserted.

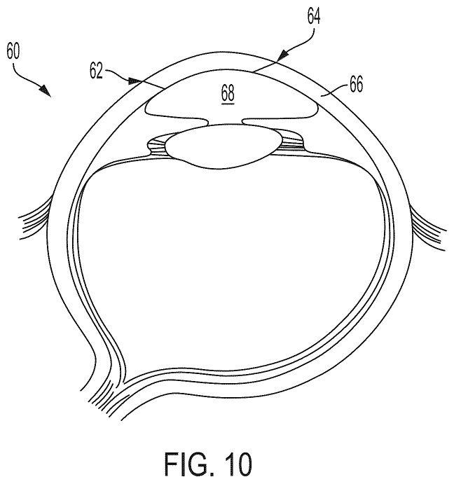

FIG. 10 is a diagrammatic sectional view of a human eye 60. Blade 12 can be used to create incisions 62, 64 in the cornea 66 and into the anterior chamber 68. An exemplary method is described with reference to FIGS. 1-10. Guard 110 is positioned on an ophthalmic instrument 10 in a partial operational position as shown in FIG. 5. With guard 110 in the partial operational position, instrument 10 is manipulated to create an incision 62, e.g., “side port” incision, in the cornea by passing first triangular portion 22 via cutting edges 22a, 22b into cornea 66. Guard 110 prevents accidentally creating a larger incision by maintaining the third portion with cutting edges 30a, 30b inside of the guard. To create the “main incision” 64 in cornea 66, guard 110 is moved to the full operational position and first triangular section 22 and posterior cutting edges 30a, 30b are used to create main incision 64. Guard 110 may be removed from instrument 10 for the full operational position. In some embodiments, guard 110 may be axially moved thereby removing blade 12 from inside of second leg 44 of the guard exposing third cutting edges 30a, 30b. Guard 110 may be rotated moving second leg 44 from directly above blade 12.

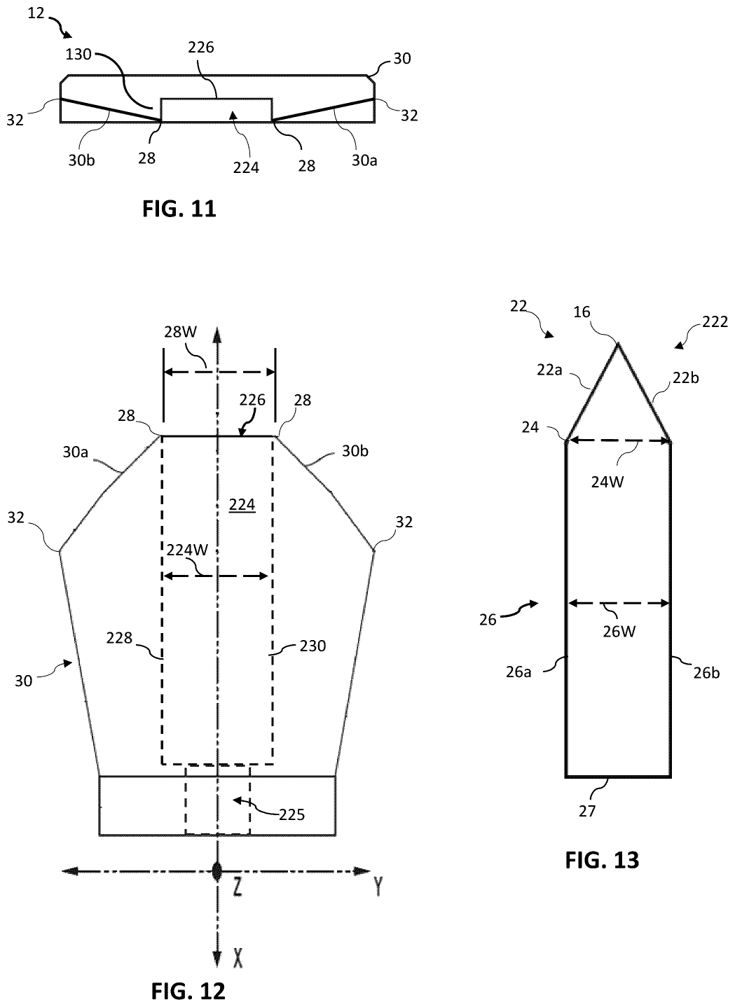

FIGS. 11-27 illustrate exemplary ophthalmic instruments 12, e.g., blades, with a central, anterior blade portion 222, that is moveable relative to a fixed posterior blade portion 30, which are described with additional reference to FIGS. 1-9. Each of the moveable central blade portion and the fixed blade portion have opposed outer cutting edges. The blade extends in a horizontal plane along a central longitudinal axis X and a lateral axis Y that is orthogonal to the longitudinal axis, blade 12 is symmetric about the central longitudinal axis.

Central, anterior blade portion 222 has a triangular head 22 on a shaft portion 26. Triangular head 22 extends from anterior tip 16 to first position 24 that has a lateral width 24W. Triangular head 22 has opposed anterior cutting edges 22a, 22b extending from anterior end 16 to first position 24. Shaft 26 has opposed parallel sides 26a, 26b and extends longitudinally from first position 24 to a terminal end 27. Shaft 26 has a lateral width 26W that is equal to or less than first lateral width 24W.

Third blade portion 30, similar to blade portion 30 in FIGS. 1-9, includes an internal pocket 224 with an opening 226 on the anterior face 130 of blade portion 30. Pocket 224 has parallel side walls 228, 230 extending longitudinally and separated by a lateral width 224W. Pocket 224 is bisected by longitudinal axis X. FIGS. 12 and 19 illustrate an exemplary pocket 224 with a hollow extension 225 extending opposite moveable blade 122. Extension 225 may exist for attaching an actuator to moveable anterior blade portion 122.

Pocket 224 is configured to moveably contain shaft portion 26 so that shaft portion 26 can be moved between a retracted position and an extended position. In the retracted position first point 24 is located in the same vertical Y-Z plane with second point 28 such that anterior cutting edges 22a, 22b and posterior cutting edges 30a, 30b are substantially continuous cutting edges. In the extended position first point 24 is separated from the second point 28 by an extension length 26L (FIGS. 15, 22). Fixed portion 30 may be constructed of one or more pieces of material.

FIGS. 11-17 illustrate embodiments where triangular base 24W and width 26W of shaft 26 are equal and substantially equal to width 28W of second position 28 where anterior cutting edges 22a, 22b and posterior cutting edges 30a, 30b present substantially continuous cutting edges. As shown in FIGS. 11 and 12, pocket width 224W is substantially equal to second width 28W.

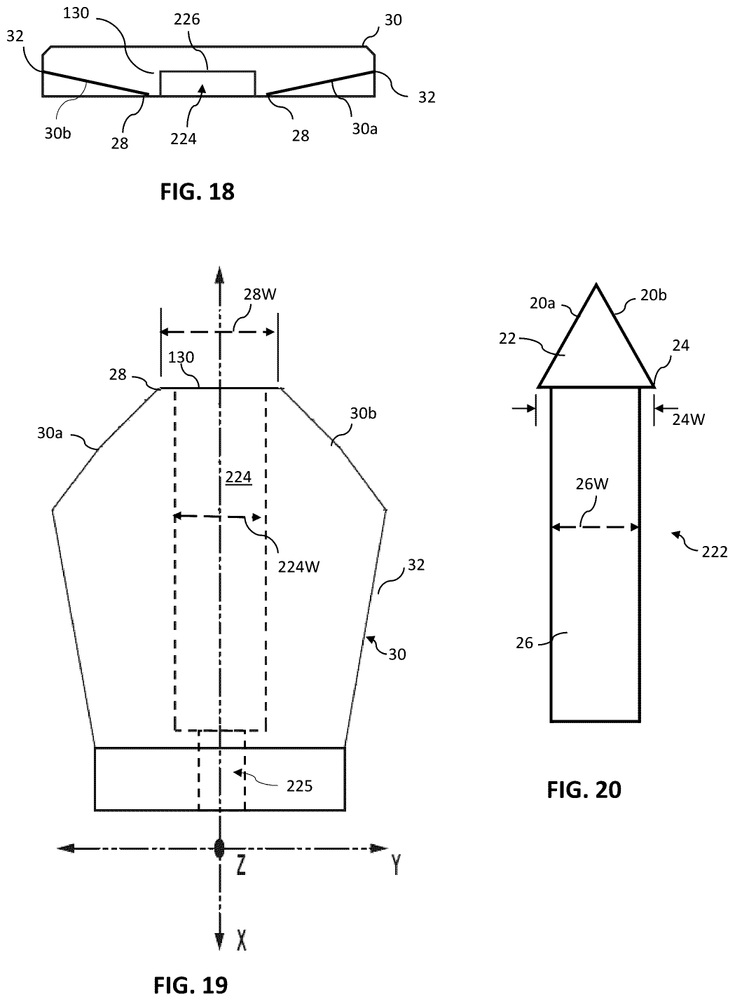

FIGS. 18-24 illustrate embodiments where anterior blade portion 222 has an arrowhead shape where triangular base width 24W is larger than shaft width 26W, shaft width 26W and pocket width 224W are less than second width 28W and less than triangular base width 24W, and triangular base width 24W may be substantially equal to second width 28W such that anterior cutting edges 22a, 22b and posterior cutting edges 30a, 30b are substantially continuous cutting edges.

FIGS. 11-13 illustrate an example moveable cutting edge blade 12 disassembled. FIGS. 11 and 12 illustrate fixed blade portion 30 and FIG. 13 illustrates anterior blade portion 222 with a triangular head 22 having a base width 24W equal to width 26W of shaft portion 26.

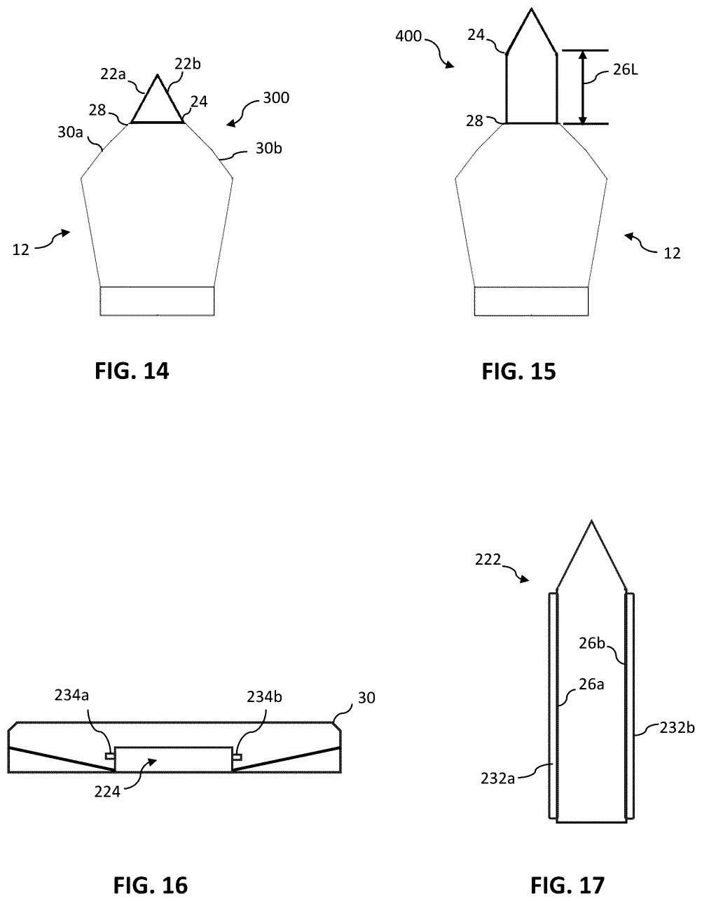

FIGS. 14 and 15 illustrate the example components in FIGS. 11-13 assembled. FIG. 14 shows blade 12 in the retracted position 300 and FIG. 15 shows blade 12 in the extended position 400. In operation, anterior cutting edge 22a and posterior cutting edge 30a present a substantially continuous cutting edge and anterior cutting edge 22b and posterior cutting edge 30b present a substantially continuous cutting edge. In retracted position 300, anterior cutting edge 22a substantially mates with posterior cutting edge 30a presenting a substantially continuous cutting edge and anterior cutting edge 22b substantially mates with posterior cutting edge 30b presenting a substantially continuous cutting edge.

FIGS. 16 and 17 illustrate another configuration of pocket 224 and anterior blade portion 222. In FIG. 17, anterior blade 222 has a tongue 232a extending on side 26a and a tongue 232b extending on side 26b. In FIG. 16, pocket 224 includes a groove 234a to dispose one of tongue 232a or tongue 232b and a groove 234b to dispose the other one of tongue 232a or tongue 232b.

FIGS. 18-24 illustrate a blade 12 where anterior blade portion 222 has an arrowhead shape where triangular base width 24W is larger than shaft width 26W, shaft width 26W and pocket width 224W are less than second width 28W and less than triangular base width 24W, and triangular base width 24W may be substantially equal to second width 28W such that in the retracted position anterior cutting edges 22a, 22b and posterior cutting edges 30a, 30b are substantially continuous cutting edges.

FIGS. 18-20 illustrate the blade with a moveable arrowhead shaped anterior blade portion disassembled. FIGS. 18 and 19 illustrate fixed blade portion 30 and FIG. 20 illustrates anterior blade portion 222 with a triangular head 22 having a base width 24W equal that is larger than width 26W of shaft portion 26. Shaft width 26W and pocket width 224W are less than second position width 28W, in this example. Triangle base width 24W is substantially equal to second position width 28W.

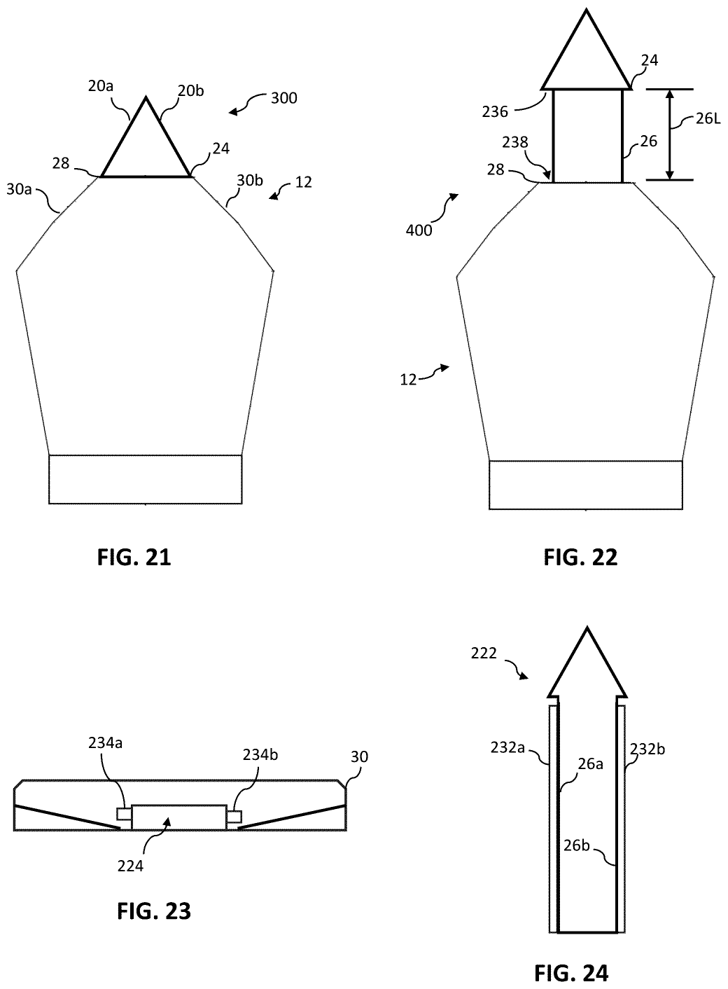

FIGS. 21 and 22 illustrate the moveable blade components in FIGS. 18-20 assembled. FIG. 21 shows blade 12 in the retracted position 300 and FIG. 22 shows blade 12 in the extended position 400. Triangular base 24 having a width that is greater than the shaft width creates a shoulder 236 that extends over and covers the interface 238 between shaft 26 and the pocket and provides, in the retracted position and the extended position, a continuous cutting edge between anterior cutting edges 22a, 22b and posterior cutting edges 30a, 30b and avoids a failure to cut between fixed cutting edges 30a, 30b and moveable cutting edges 22a, 22b.

In retracted position 300, anterior cutting edge 22a and posterior cutting edge 30a mate, or substantially mate, presenting a substantially continuous cutting edge. Similarly, anterior cutting edge 22b and posterior cutting edge 30b mate, or substantially mate, presenting a substantially continuous cutting edge.

FIGS. 23 and 24 illustrate another configuration of pocket 224 and arrowhead shaped anterior blade portion 222. In FIG. 23, anterior blade 222 has a tongue 232a extending on side 26a and a tongue 232b extending on side 26b. In FIG. 24, pocket 224 includes a groove 234a to dispose one of tongue 232a or tongue 232b and a groove 234b to dispose the other one of tongue 232a or tongue 232b.

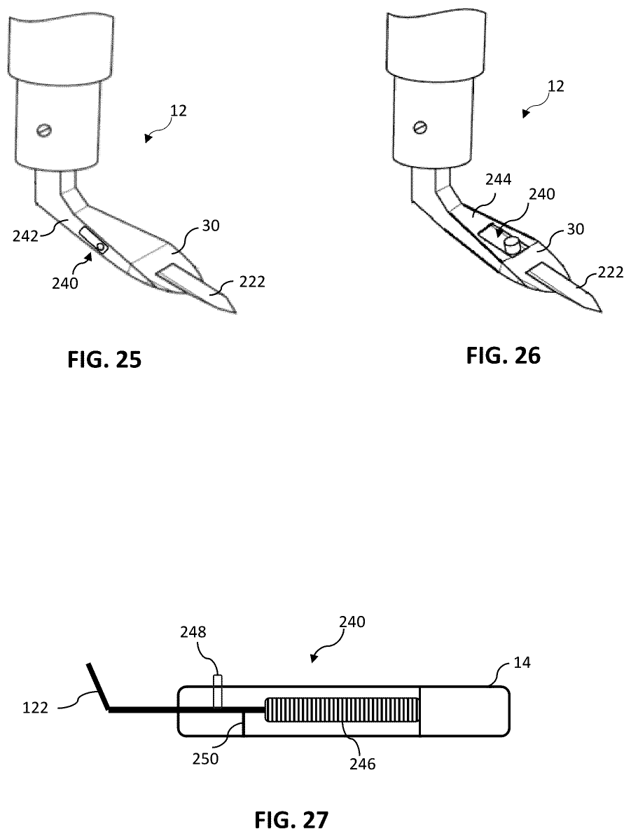

FIGS. 25-27 illustrate examples of ophthalmic instrument 12 with an actuator 240 to move anterior blade portion 222 relative to fixed blade portion 30. FIG. 25 illustrates an example of actuator 240 accessible through a lateral side 242 of fixed blade portion 30. FIG. 26 illustrates an example of actuator 240 accessible through a top side 244 of fixed blade portion 30. With reference to FIG. 27, actuator 240 may include, for example, a biasing element 246, e.g., a spring, to urge moveable blade portion 122 to a failsafe position. Biasing mechanism 246 may urge moveable blade portion 122 to the retracted position or the extended position. Actuator 240 may include a switch 248, e.g., tang, fixed with the moveable blade portion 122 for a person to manually move the moveable blade portion against biasing element 246 into a lock position where a latch 250 secures the moveable blade portion. The actuator, or portions of the actuator, may be located in blade 12 and/or the handle 14. For example, extension passage 225 in FIGS. 12 and 19 may provide access for attaching the actuator to the moveable blade portion.

Although relative terms such as “outer,” “inner,” “upper,” “lower,” and similar terms have been used herein to describe a spatial relationship of one element to another, it is understood that these terms are intended to encompass different orientations of the various elements and components in addition to the orientation depicted in the figures. Furthermore, as used herein, the terms “connect,” “connection,” “connected,” “in connection with,” and “connecting” may be used to mean in direct connection with or in connection with via one or more elements. Similarly, the terms “couple,” “coupling,” and “coupled” may be used to mean directly coupled or coupled via one or more elements. The terms “substantially,” “approximately,” “generally,” and “about” are defined as largely but not necessarily wholly what is specified (and includes what is specified; e.g., substantially 90 degrees includes 90 degrees and substantially parallel includes parallel), as understood by a person of ordinary skill in the art. The extent to which the description may vary will depend on how great a change can be instituted and still have a person of ordinary skill in the art recognized the modified feature as still having the required characteristics and capabilities of the unmodified feature.

The foregoing outlines features of several embodiments so that those skilled in the art may better understand the aspects of the disclosure. Those skilled in the art should appreciate that they may readily use the disclosure as a basis for designing or modifying other processes and structures for carrying out the same purposes and/or achieving the same advantages of the embodiments introduced herein. Those skilled in the art should also realize that such equivalent constructions do not depart from the spirit and scope of the disclosure and that they may make various changes, substitutions, and alterations herein without departing from the spirit and scope of the disclosure. The scope of the invention should be determined only by the language of the claims that follow. The term “comprising” within the claims is intended to mean “including at least” such that the recited listing of elements in a claim are an open group. The terms “a,” “an” and other singular terms are intended to include the plural forms thereof unless specifically excluded.

What is claimed is:

1. An ophthalmic instrument, comprising:
a blade extending in a horizontal plane along a central longitudinal axis and a lateral axis orthogonal to the longitudinal axis, the blade symmetric about the central longitudinal axis, wherein the blade comprises:
an anterior blade portion having a triangular head and a shaft;
the triangular head having opposed anterior cutting edges that extend from an anterior tip to a base position, the base position having a base width parallel with the lateral axis;
the shaft having a shaft width between opposed shaft edges that is equal to the base width, the shaft extending aft from the base position;
a fixed blade portion having a forward lateral position with a forward width and a posterior lateral position with a posterior width that is greater than the forward width and opposed posterior cutting edges that extend from the forward lateral position to the posterior lateral position; and
a pocket formed in the fixed blade portion extends aft along, and bisected by, the central longitudinal axis:
wherein the shaft is positioned in the pocket and moveable between a retracted position with the base position located with the forward lateral position whereby the opposed anterior cutting edges and the opposed posterior cutting edges present substantially continuous cutting edges, and an extended position where the base position is spaced forward of the forward lateral position by an extension length.

2. The ophthalmic instrument of claim 1, further comprising an actuator coupled with the anterior blade portion, the actuator having a biasing element urging the anterior blade portion to one of the retracted position or the extended position.

3. The ophthalmic instrument of claim 1, further comprising an actuator coupled with the anterior blade portion, the actuator having a biasing element urging the anterior blade portion to the retracted position.

4. The ophthalmic instrument of claim 3, wherein the actuator comprises:
a piece fixedly coupled to the anterior blade portion; and
a latch to secure the piece in a stationary position when the anterior blade portion is in the extended position.

5. The ophthalmic instrument of claim 4, wherein the actuator is positioned in a handle attached to the fixed blade portion.

6. The ophthalmic instrument of claim 4, wherein the actuator is positioned in the blade.

7. The ophthalmic instrument of claim 1, wherein the pocket has a pocket width that is substantially equal to the forward width of the forward lateral position.

8. The ophthalmic instrument of claim 7, further comprising an actuator coupled with the anterior blade portion, the actuator having a biasing element urging the anterior blade portion to one of the retracted position or the extended position.

9. The ophthalmic instrument of claim 7, further comprising an actuator coupled with the anterior blade portion, the actuator having a biasing element urging the anterior blade portion to the retracted position.

10. An ophthalmic instrument, comprising:
a blade extending in a horizontal plane along a central longitudinal axis and a lateral axis orthogonal to the longitudinal axis, the blade symmetric about the central longitudinal axis, wherein the blade comprises:
an anterior blade portion having a triangular head and a shaft;
the triangular head having opposed anterior cutting edges that extend from an anterior tip to a base position, the base position having a base width parallel with the lateral axis;
the shaft having a shaft width between opposed shaft edges that is less than the base width, the shaft extending aft from the base position;
a fixed blade portion having a forward lateral position with a forward width and a posterior lateral position with a posterior width that is greater than the forward width and opposed posterior cutting edges extending from the forward lateral position to the posterior lateral position; and
a pocket formed in the fixed blade portion and extending aft along and bisected by the central longitudinal axis:
wherein the shaft is positioned in the pocket and moveable between a retracted position with the base position located with the forward lateral position whereby the opposed anterior cutting edges and the opposed posterior cutting edges present substantially continuous cutting edges, and an extended position where the base position is spaced forward of the forward lateral position by an extension length.

11. The ophthalmic instrument of claim 10, wherein the pocket has a pocket width that is less than the forward width of the forward lateral position.

12. The ophthalmic instrument of claim 10, wherein the base position overlaps an interface between the shaft and the pocket.

13. The ophthalmic instrument of claim 10, wherein the base width is substantially equal to the forward width of the forward lateral position.

14. The ophthalmic instrument of claim 10, wherein:
wherein the pocket has a pocket width that is less than the forward width of the forward lateral position; and
the base width is substantially equal to the forward width of the forward lateral position.

15. The ophthalmic instrument of claim 10, further comprising an actuator coupled with the anterior blade portion, the actuator having a biasing element urging the anterior blade portion to one of the retracted position or the extended position.

16. The ophthalmic instrument of claim 15, wherein:
wherein the pocket has a pocket width that is less than the forward width of the forward lateral position; and
the base width is substantially equal to the forward width of the forward lateral position.

17. The ophthalmic instrument of claim 10, further comprising an actuator coupled with the anterior blade portion, the actuator having a biasing element urging the anterior blade portion to the retracted position.

18. The ophthalmic instrument of claim 17, wherein:
wherein the pocket has a pocket width that is less than the forward width of the forward lateral position; and
the base width is substantially equal to the forward width of the forward lateral position.
